(19) United States
(12) Patent Application Publication
(10) Pub. No.: US 2006/0052706 A1
(43) Pub. Date: Mar. 9, 2006

(54) Phased array ultrasound for cardiac ablation

(76) Inventors: Kullervo Hynynen, Medfield, MA (US); Laurence Epstein, Medfield, MA (US); Ferenc A. Jolesz, Brookline, MA (US)

Correspondence Address: FISH & RICHARDSON PC, P.O. BOX 1022, MINNEAPOLIS, MN, 55440-1022, US

(21) Appl. No.: 11/208,353

(22) Filed: Aug. 19, 2005

Related U.S. Application Data
(60) Provisional application No. 60/603,050, filed on Aug. 20, 2004.

(51) Int. Cl. A61B 8/14

(52) U.S. Cl. 600/459

(57) ABSTRACT

A system is provided for performing ablation of target tissue. The system includes an ultrasound phased array having a plurality of ultrasonic transducers and drive circuitry that is configured to generate signals that cause the ultrasonic transducers to focus ultrasound radiation at the target tissue. The system also includes a diagnostic system configured to collect diagnostic data that is indicative of a condition of the target tissue and computational circuitry that is configured to control the drive circuitry based on the diagnostic data.

### CROSS-RELATED APPLICATION

Under 35 U.S.C. 119(e)(1), this application claims the benefit of provisional application serial number, 60/603,050, filed Aug. 20, 2004.

### TECHNICAL FIELD

The present invention relates to medical applications of ultrasound and in particular, to ultrasound for cardiac ablation.

### BACKGROUND

Cardiac arrhythmias are characterized by erratic cardiac contractions. These erratic contractions are often confined to areas of the cardiac muscle that have abnormal electric conduction, refractoriness, or impulse formation. These abnormalities disturb the normal propagation of the electric signals through the muscle resulting in abnormal muscle contraction. A variety of surgical and non-surgical treatments are available for cardiac arrhythmias. The non-surgical treatments are anti-arrhythmetic drugs designed to alter the electrophysiologic properties of the cardiac tissue. Though these drugs decrease the likelihood that an arrhythmia will occur, their efficacy is limited. Moreover, they have potentially fatal side effects. For these reasons, pharmacological approaches for treating cardiac arrhythmia have been widely supplanted by surgical approaches that irreversibly damage or ablate the tissue regions that cause and sustain the arrhythmias.

Over the past two decades open-heart ablative surgery has been replaced by catheter cardiac ablation, a minimally-invasive procedure in which a catheter is inserted transcutaneously into an artery or a vein and guided fluoroscopically to the heart. The catheter delivers energy to the problematic site. This energy heats the arrhythmogenic tissue until it coagulates, thus destroying the tissue. Radio frequency (RF) energy is most commonly used, though a variety of energy sources, including direct currents, microwaves, cryothermic sources, and lasers can be used for ablating tissue.

Although catheter ablation has become a standard form of treatment, it has major limitations in both efficacy and safety. Conventional catheter cardiac ablation techniques are limited in their ability to accurately identify arrhythmogenic tissue. In addition, catheter cardiac ablation has great difficulty producing deep transmural, continuous lesions. The invasive nature of catheter cardiac ablation can lead to significant complications including severe pain, adverse drug reaction from anesthesia, infection, thrombophlebitis, myocardial infarction, perforation, hemopericardium, and cardiac tamponade that can ultimately prove fatal. Furthermore, catheter cardiac ablation is performed under fluoroscopy guidance, a procedure that emits ionizing radiation. If the procedure is prolonged, the patient and the physician are at risk for sustaining a hazardous level of exposure.

### SUMMARY

In an aspect, the invention features a system for performing ablation of target tissue. The system includes an ultrasound phased array having a plurality of ultrasonic transducers and drive circuitry coupled to the ultrasonic transducers. The drive circuitry is configured to generate signals that cause the ultrasonic transducers to focus ultrasound radiation at the target tissue. The system also includes a diagnostic system configured to percutaneously collect diagnostic data that is indicative of a condition of the target tissue and computational circuitry that is interfaced to the drive circuitry and to the diagnostic system. The computational circuitry is configured to control the drive circuitry based on the diagnostic data.

In some embodiments, the ultrasound phased array is two-dimensional and configured for placement in a patient's esophagus. In some embodiments, the drive circuitry includes multi-channel radio-frequency drivers and the diagnostic system includes an imaging system for producing an image of the target tissue. Examples of the imaging system include a magnetic resonance imaging (MRI) system, an ultrasound imaging system, a computed tomography imaging system, an x-ray imaging system, and a positron-emission tomography imaging system. In some embodiments the diagnostic system includes a temperature monitoring system which may, for example, include an MRI system, and is configured to collect the diagnostic data in real-time.

In another aspect, the invention features methods and computer readable mediums for performing ablation of target tissue. The method includes identifying a target location of the target tissue from an image of the target tissue; focusing ultrasound radiation from an ultrasound phased array at the target location; collecting diagnostic data percutaneously, the diagnostic data being indicative of a condition of the target tissue; and controlling a characteristic of the ultrasound radiation (e.g., phase, frequency, and power) based on the diagnostic data such that the ultrasound radiation ablates the target tissue without damaging surrounding tissue. The computer readable medium includes instructions for performing the method.

In some embodiments, the target location is determined in relation to a periodic triggering event (e.g., a heartbeat) and the ultrasound radiation is focused at the target location for a predefined period of time in response to detecting the triggering event. In other embodiments the target location is determined in real-time. In some embodiments the coordinates of the image are transformed to coordinates of the ultrasound phased array. In some embodiments collecting diagnostic data also includes acquiring temperature data that is indicative of ablation.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows a system block diagram for an ultrasonic ablation system;

FIG. 2A shows a two-dimensional ultrasound phased array from the system of FIG. 1;

FIG. 2B shows a two-dimensional ultrasound phased array from the system of FIG. 1;

FIG. 3 shows a one-dimensional ultrasound array from the system of FIG. 1;

FIG. 4 depicts an interface between the transducers in the array and the drive circuitry in the system of FIG. 1;

FIG. 5 depicts ablation using the two-dimensional ultrasound array of FIG. 2A;

FIG. 6 shows the system of FIG. 1 deployed for trans-esophageal cardiac ablation;

FIG. 7 illustrates the functions carried out by the computer system from the system of FIG. 1;

FIG. 8 illustrates the coordinate registration function shown in FIG. 7;

FIG. 9 illustrates target-tissue identification and motion detection shown in FIG. 7;

FIG. 10 illustrates continuous ablation shown in FIG. 7;

FIG. 11 illustrates periodic ablation shown in FIG. 7;

FIG. 12 illustrates image-array coordinate registration;

FIG. 13 illustrates sonication volume movement detection;

FIG. 14 shows a diagram of a simulation configuration showing an ultrasound phased array, esophageal wall and the three groups of foci in cardiac muscle;

FIG. 15 shows plots of a non-uniform grid and a simulated ultrasound beam;

FIG. 16 shows plots of squared acoustic pressure amplitudes that result from sonicating nine of the foci shown in FIG. 15;

FIGS. 17-19 show plots of lesions that resulted from sonicating the first group of foci, shown in FIG. 15, over different sonication durations;

FIGS. 20A and 20B show plots safe and unsafe sonications that were simulated for the foci shown in FIG. 15;

FIG. 21 shows a plot of average transducer acoustic power that was achieved at various sonication durations for different peak temperatures; and

FIG. 22 shows a plot of the average length and width of the simulated lesions that were achieved at various sonication durations for different peak temperatures.

### DETAILED DESCRIPTION

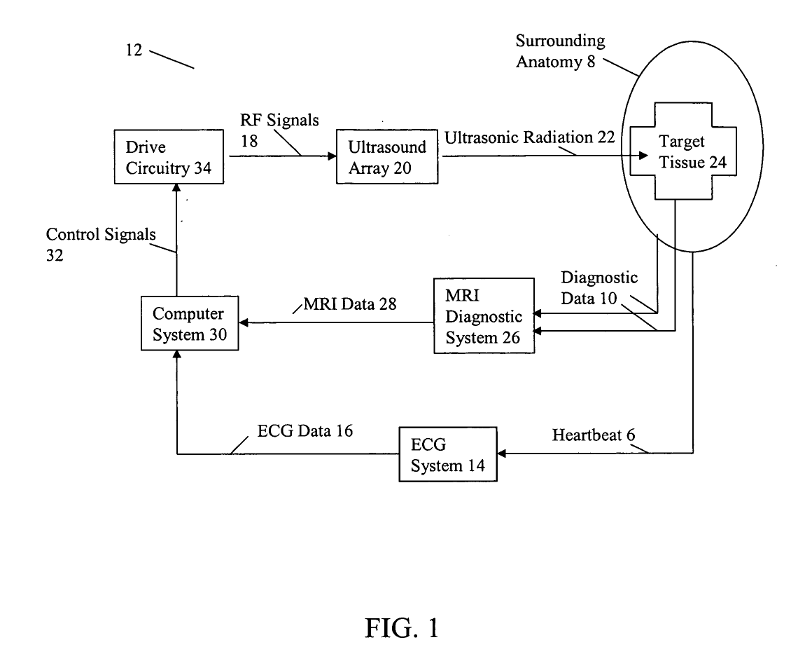

FIG. 1 depicts a block diagram of the ultrasound ablation system 12 for performing ablation on a target tissue 24 using ultrasonic radiation 22. The system includes an ultrasound array 20 directed toward the target tissue 24, drive circuitry 34 for driving the array, an magnetic-resonance imaging (MRI) diagnostic system 26 for receiving data indicative of the condition of the tissue, an optional (electrocardiogram) ECG system 14 for receiving vital sign information 6 from the patient, and a computer system 30 for controlling the drive circuitry 34 in response to data provided by the MRI diagnostic system 26 and the ECG system 14. Other types of diagnostic tools such as ultrasonic imaging, computed tomography imaging (CT scan), x-ray, positron-emission tomography imaging (PET scan) could be used in place of the MRI diagnostic system 26. Moreover any combination of diagnostic tools could also be used. In addition to the ECG system 14, other types of vital-sign monitoring systems such as electroencephalogram (EEG), and ultrasonic imaging could be used.

The MRI diagnostic system 26 collects diagnostic data 10 from the target tissue 24 and the surrounding anatomy 8 of the patient. The diagnostic data is collected percutanously, i.e., without puncturing or breaking the skin. In one implementation, the MRI diagnostic system 26 acquires an image on a line-by-line basis using fast line-scan MRI. The fast line-scan MRI technique enables the image data in a line to be analyzed in real-time as the line is scanned without waiting for the whole scan to finish. The MRI diagnostic system 26 processes the diagnostic data 10 into a series of images and temperature measurements that collectively comprise MRI data 28. This MRI data 28, and optimally, ECG data 16, is sent to a computer system 30.

From the MRI data 28 and the ECG data 16, the computer system 30 identifies the location of the target tissue 24 as a function of time. The computer system 30 could also identify the location of the target tissue in real-time. The computer system 30 calculates the radio-frequency (RF) signals 18 required to focus the ultrasonic radiation 22 at the target-tissue location and sends information representative of those RF signals 18 as control signals 32 to the drive circuitry 34. In response to the control signals 32, the drive circuitry 34 generates radio frequency (RF) signals 18 that cause the ultrasound array 20 to focus ultrasonic radiation 22 at the target-tissue location. While the tissue is undergoing sonication, the MRI diagnostic system 26 sends a stream of MRI data 28 to the computer system 30. Using the MRI Data 28, the computer system 30 monitors the movement of the target tissue, tracks the progress of the sonication, and determines when the ablation is complete. In addition to MRI, other imaging techniques such as ultrasound echo imaging could be used to track the target-tissue movement and monitor the sonication.

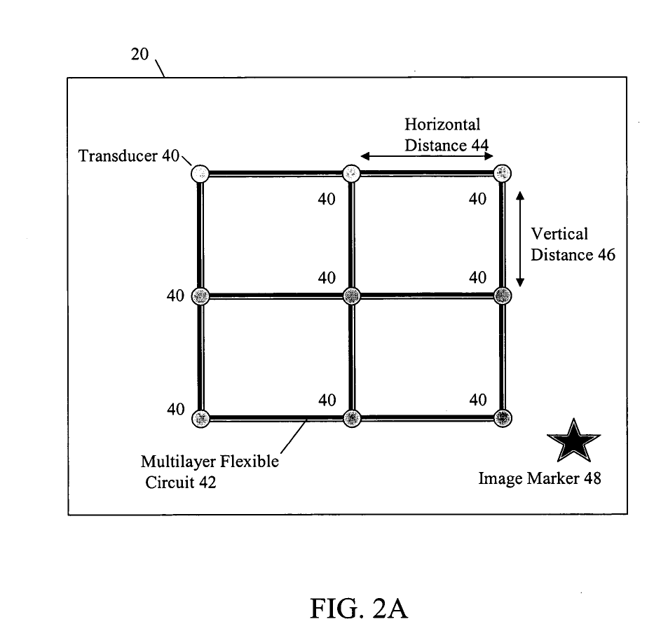

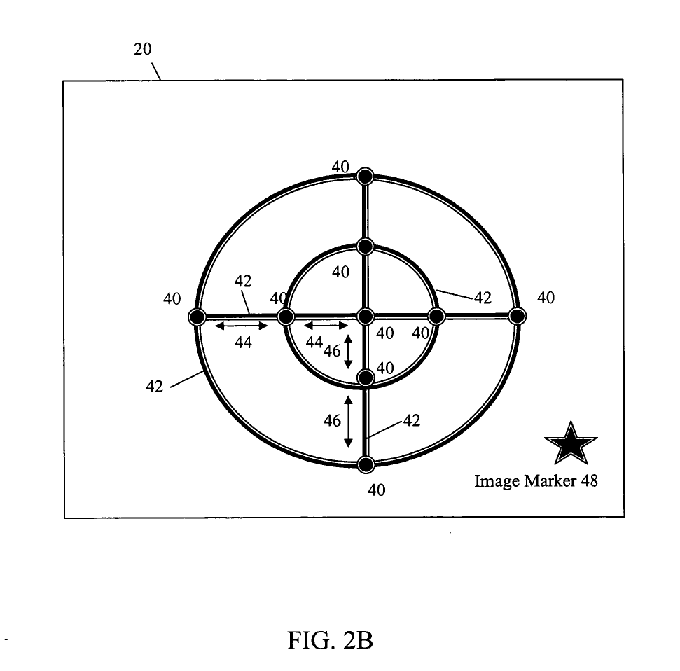

Referring to FIGS. 2A and 2B, in one embodiment, shown in FIGS. 2A and 2B, the array 20 is a two-dimensional ultrasound array having multiple transducers 40 and an image marker 48. The transducers 40 are connected together by multilayer flexible circuits 42, though other types of connective circuits, such as micro-coaxial cables, could also be used. Any number of transducers could be arranged in any pattern, though a preferred arrangement has a thousand transducers arranged in concentric circles in which the horizontal distances 44 and vertical distances 46 between the transducers are equal to half the wavelength of the ultrasonic radiation 22. The vertical and horizontal spacing between transducers could be equal but different than one half wavelength of the ultrasonic radiation or they could be unequal. Other patterns could include a grid, a spiral, or an irregular pattern, though any pattern is possible. The array 20 could include transducers 40 of the same size, shape, and material composition or the array 20 could include any combination of transducers 40 of different sizes, shapes, and material compositions.

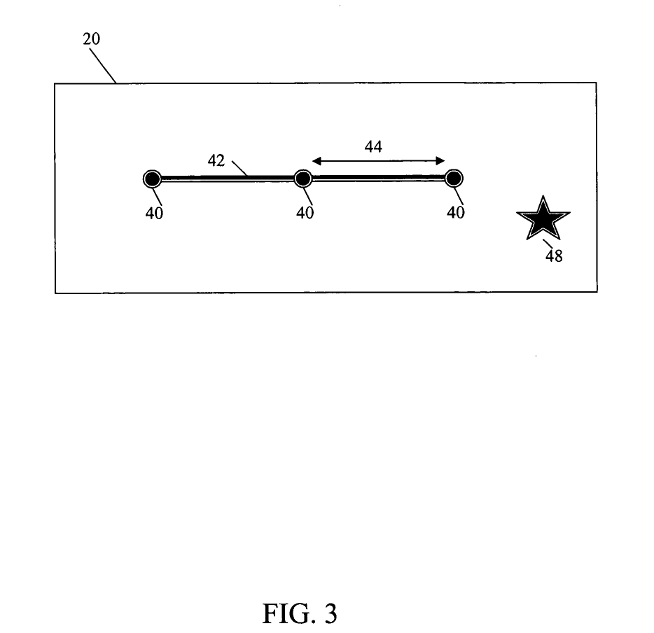

As seen in FIG. 3, the ultrasound array 20 could also be a one-dimensional array. However, a two-dimensional array is preferred over a one-dimensional array because the focus 62 of the ultrasonic radiation 22 produced by a two-dimensional array can be moved freely in three dimensions. For a one-dimensional array, the focus is limited to the axial plane that extends perpendicular to the array plane, hence to enable angular positioning of the focus 62 the one-dimensional array would need to be mechanically rotated.

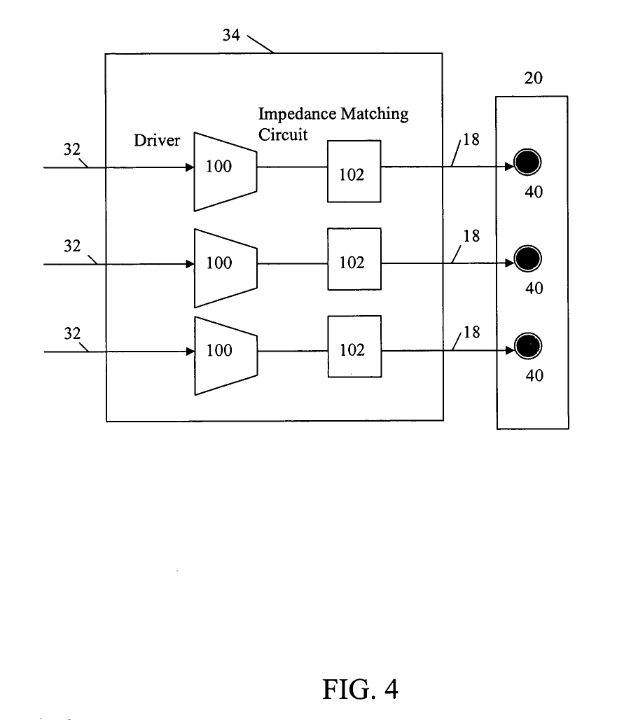

FIG. 4 shows the interface between the drive circuitry 34 and the array 20. Each transducer 40 in the ultrasound array 20 is connected to a driver 100 through an impedance matching circuit 102. Each driver 102 includes a dedicated signal generator and an amplifier. The driver 100 receives a control signal 32 and produces an RF signal 18 as a response. The RF signal 18 in turn stimulates an associated transducer 40 to produce ultrasonic radiation of a required frequency and phase. The drive circuitry 34 is capable of driving thousands of transducers 40. For such a large number, individual electrical impedance matching of each transducer 40 to the output impedance of its corresponding driver 100 may not be feasible. A more practical approach is to sample the output impedances of the drivers and to provide an impedance matching circuit that gives the best matching when duplicated for all array elements. This type of matching circuit could be designed on a circuit board and mass-produced.

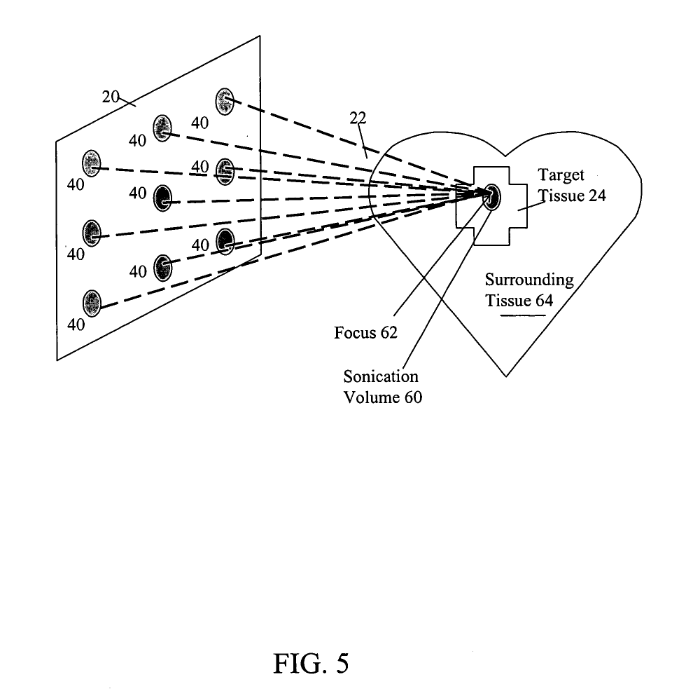

FIG. 5 shows the ablation of the target tissue 24 at a sonication volume 60. The sonication volume 60 is the volume of target tissue 24 that is ablated at the focus 62. If the focus 62 is not large enough to cover an entire volume of target tissue 24, the target-tissue volume is divided into a series of contiguous sonication volumes that are ablated individually. The ultrasonic radiation 22 is chosen to be sufficient to ablate the sonication volume 60 within the target tissue 24 without damaging any of the surrounding tissue 64.

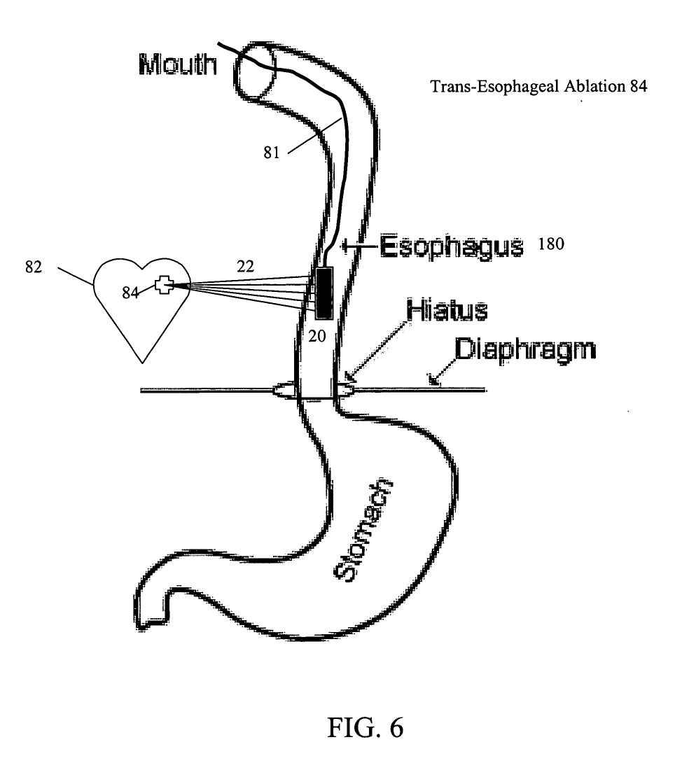

FIG. 6 shows how the ablation system 12 could be used to perform trans-esophageal cardiac ablation. In this procedure, the phased array is positioned in the esophagus 80 of the patient. Ultrasonic radiation 22, delivered to the heart from an array 20 inserted into the esophagus 80 by a catheter 81, ablates arrhythmogenic target tissue 84 without damaging the esophagus 80 or any surrounding heart tissue 82. In addition to the esophagus, the array could be inserted into other lumens, such as those associated with the colon, nose, and ears, or the array could be placed external to the body. The target tissue 24 could include any type of abnormal tissue, such as cancerous and benign tumors, fibroid cysts, polyps, and infected tissue.

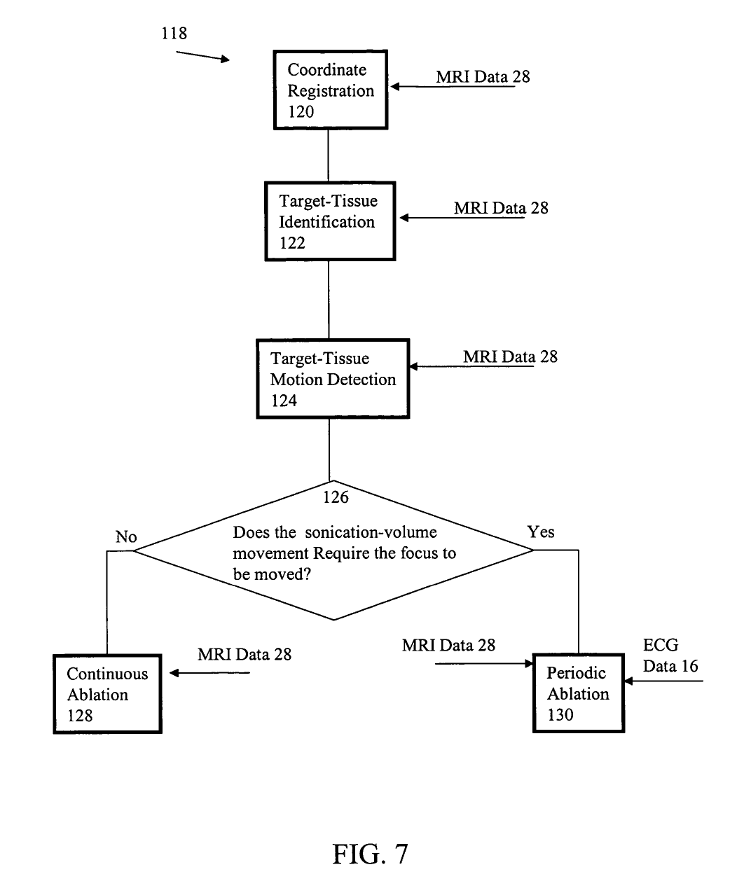

FIG. 7 depicts a block diagram of the computer system functions 118 performed by the computer system 30. The functions 118 include a coordinate registration function 120, a target-tissue identification function 122, a target-tissue motion detection function 124, a decision function 126, a continuous ablation function 128, and periodic ablation function 130. The functions 118 receive MRI data 28. The periodic ablation function 130 also receives ECG data 16.

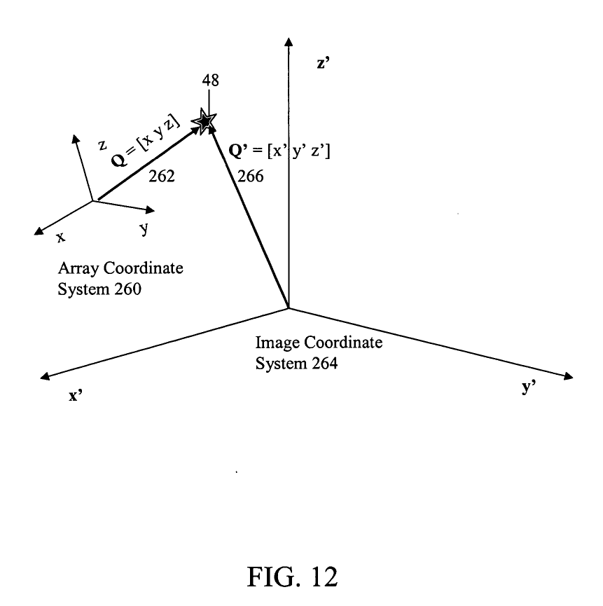

The ablation process begins when the coordinate registration function 120 registers the coordinates of the ultrasound array 20 with the coordinates of the MRI data 28. Then, the target-tissue identification function 122 identifies the image space coordinates of the target tissue. These coordinates are translated from image space 264 to array space 260 (see FIG. 12) by the coordinate registration function 120. The target-tissue motion detection function 124 determines how much a sonication volume 60 on the target tissue 24 is moving. A decision function 126 then determines, on the basis of this motion, to pursue either continuous ablation or periodic ablation.

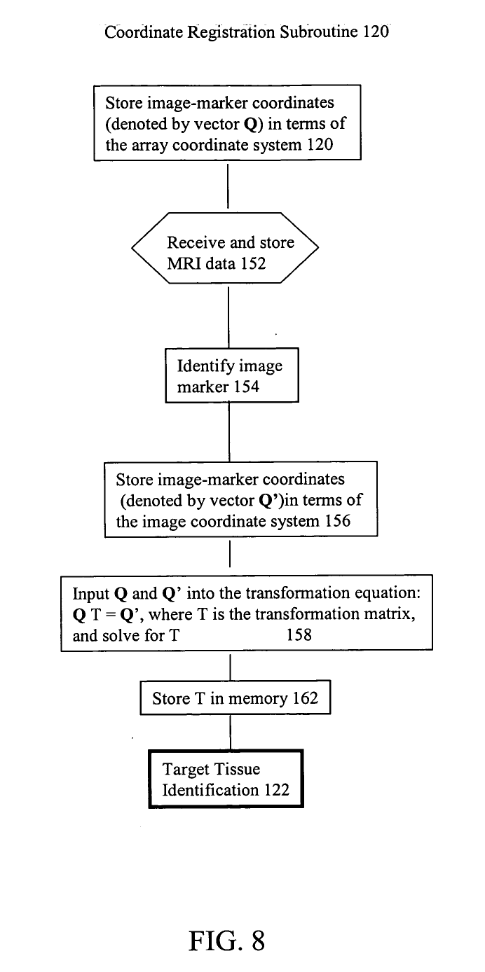

FIG. 8 shows the coordinate registration function 120 of FIG. 7 in more detail. As shown in FIG. 12, an image marker 48 has known coordinates 262 denoted by Q, where Q=[x y z] is referenced to the array-coordinate system. The coordinate registration function 120 begins by storing the coordinates 262 in memory (step 150). An MRI image is taken of the image marker 48 and its surroundings. The computer system 30 receives and stores the resulting MRI data (step 152) and identifies the image marker (step 154). The image marker 48 is made of a material that makes it easily identifiable in an MRI image. The identification step (step 154) could be performed using an image segmentation algorithm. The coordinates 156 of the marker image (denoted by Q) are then read from the image and stored (step 156). The transformation matrix T that relates the array-coordinate system to the image-coordinate system is determined by solving the transformation equation: Q T=Q′ (step 158). The transformation matrix T is then stored in memory (step 162) to be used in subsequent functions for transforming image-coordinates into array-coordinates.

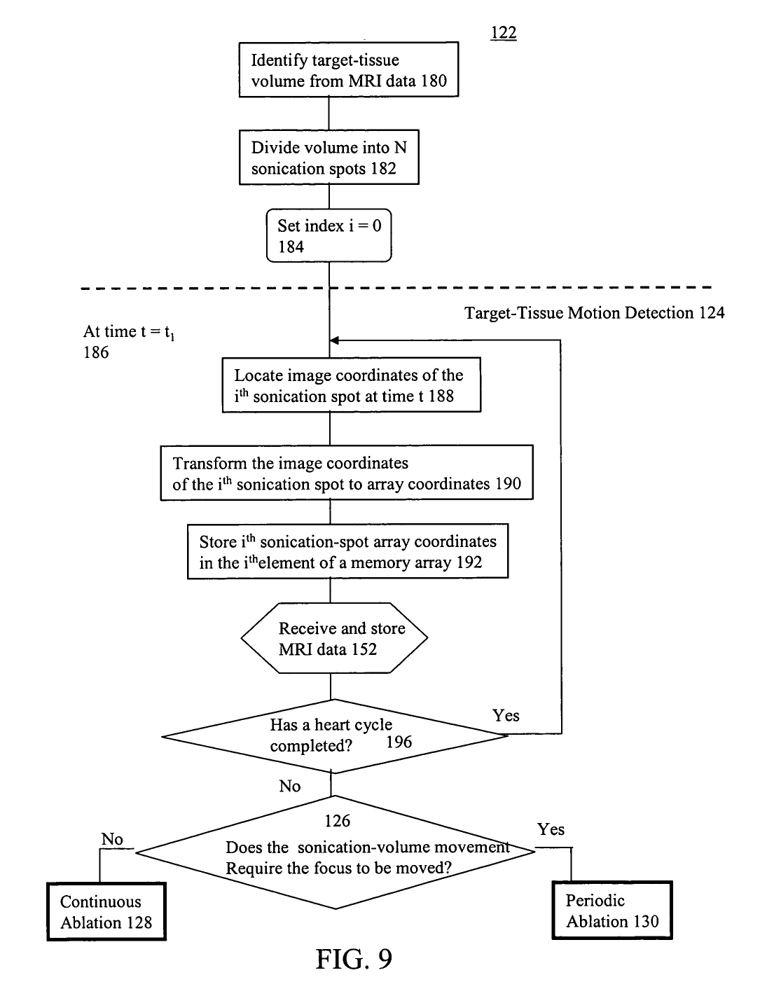

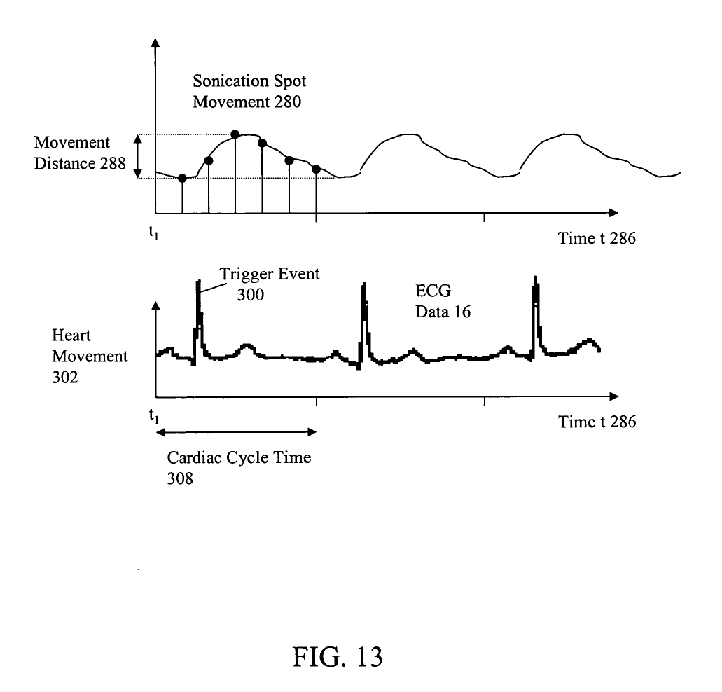

FIG. 9 depicts the target-tissue identification function 122 and target-tissue motion detection function 124 of FIG. 7 in more detail. From the MRI data 28, the target-tissue volume is identified (step 180). The identification could be performed using an image segmentation algorithm. The image is then divided into a number N of sonication volumes 60 (step 182). At any given time t₁ 186, image coordinates of the first sonication volume (i=0) 184 are located (step 188), transformed into array coordinates (step 190), and stored in a memory array (step 192). As shown in FIG. 13, the coordinates of the first sonication volume are determined at subsequent times over a cardiac cycle period 308. By calculating the maximum distance 288 between the sets of coordinates, the sonication volume movement 280 is determined for the duration of a cardiac cycle 196. If the movement of the sonication volume requires that the focus be moved (step 200), the periodic ablation function 130 is executed; otherwise the continuous ablation function 128 is executed.

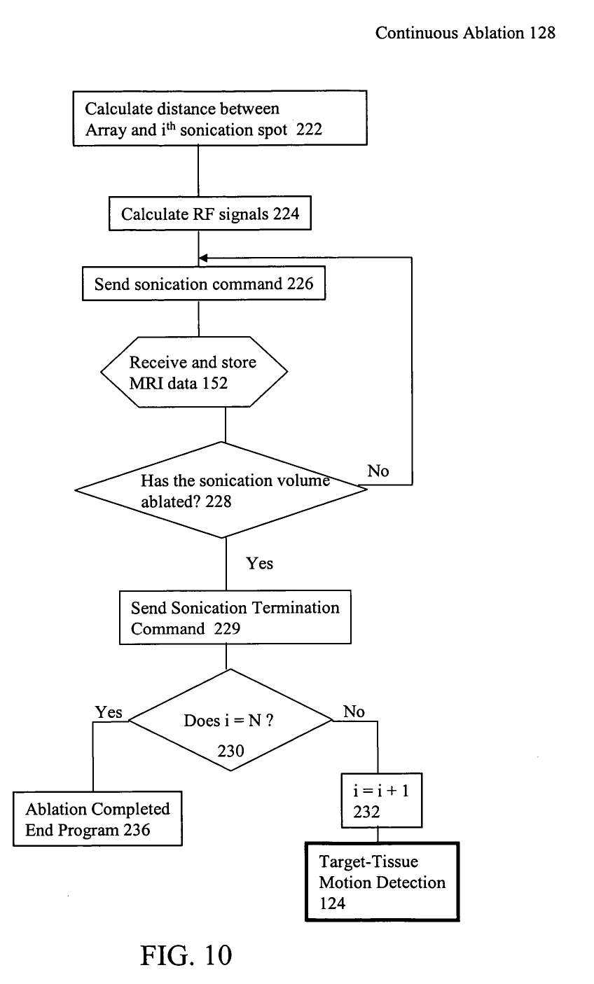

FIG. 10 illustrates continuous ablation 128. The distance between the sonication volume and the array is calculated (step 222). The computer system 30 then calculates the RF signals required to cause the array 20 to focus the ultrasonic radiation 22 at the sonication-volume coordinates (step 224). The computer system 30 then sends a sonication command to the drive circuitry 34 (step 226). MRI data is received and stored (step 152). On the basis of this data, the system 30 determines if the target tissue 24 at the sonication volume 60 has ablated. If not, the system 30 sends another sonication command. This procedure continues until ablation has occurred. When the sonication is complete, the system moves to the next sonication volume and continues until all target tissue sonication volumes have been ablated (steps 230, 232, 236).

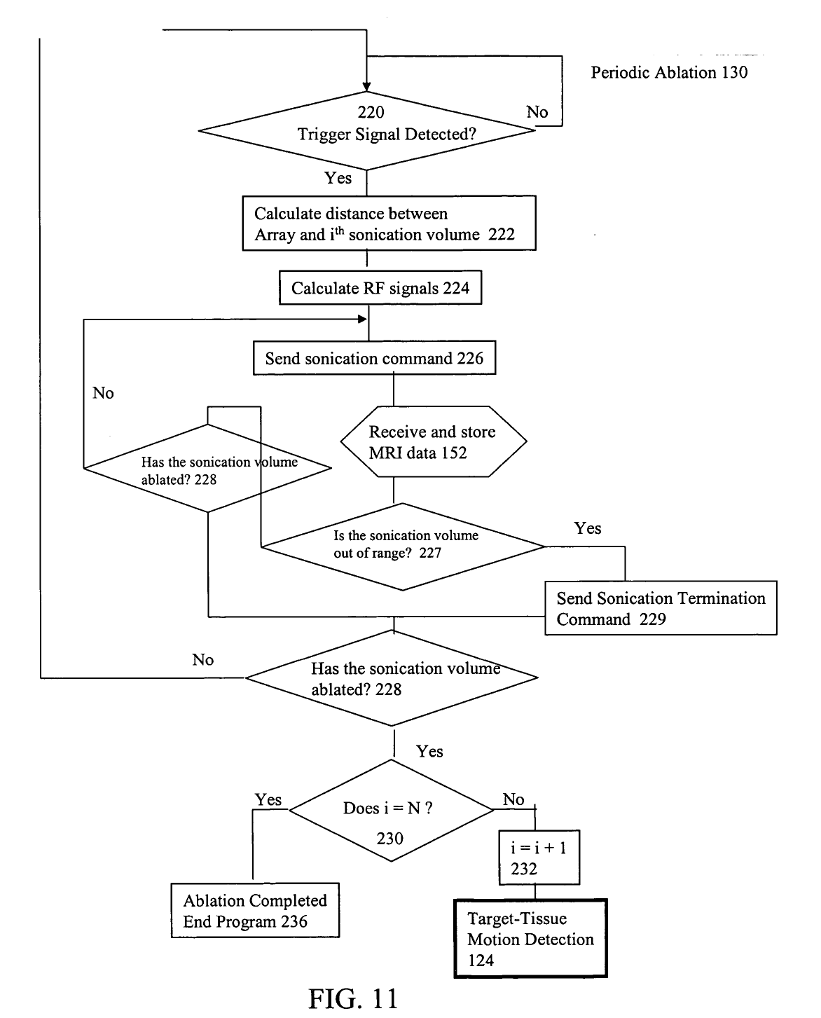

In FIG. 11, the periodic ablation function 130 proceeds in much the same way as the continuous ablation function 128 except that the sonications are initiated when a trigger event 300 is detected (step 220). The trigger event could be the peak amplitude of an ECG signal shown in FIG. 13. Once the trigger event is detected, the system 30 performs the necessary calculations and sends a sonication command signal to the drivers to initiate sonication (steps 222, 224, 226). During the sonication, MRI data is received and stored (step 152). The system uses this data to determine if the sonication volume 60 is still in the focus or if it has moved out of range (step 227). While the sonication volume 60 is still in range of the focus 62, the system 30 will monitor its temperature to determine if it has ablated (step 228). If the sonication volume 60 has moved outside the range of the focus 62, the system 30 will send a command to terminate the sonication. If the sonication volume 60 is not fully ablated, the system 30 will have to wait until it detects another trigger event before it can resume sonication. This procedure continues until ablation has occurred. When the sonication is complete, the system moves to the next sonication volume and continues until all target-tissue sonication volumes have been ablated (steps 230, 232, 236).

The procedures for performing continuous ablation and periodic ablation are not limited to those illustrated in FIG. 10 and FIG. 11, respectively. The location of the target-tissue as a function of time could be measured using imaging methods other than MRI. The information could be stored over several heart cycles. In a procedure for continuous ablation, the phased-array would then focus the radiation according to this predetermined pattern using the heartbeat as a reference. Another possible method includes providing online location feedback from MRI line-scan image data. Instead of using MRI to provide the feedback data, ultrasound could be used. Some of the transducers in the phased-array could be used for sending diagnostic ultrasound pulses and receiving the echoes to locate the sonication volume.

### EXAMPLES

The feasibility of transesophageal cardiac thermal ablation using a planar ultrasound two-dimensional phased array was investigated in computer simulation studies.

Summary of Results

The results of the study showed that by varying sonication duration and power, the array can produce controllable tissue coagulation without damage to the overlaying or surrounding tissues. The array modeled in the studies was a two-dimensional planar ultrasound phased array having a 1 MHz output frequency, dimensions of 60×10 mm², 0.525 mm inter-element spacing, and 114×20 transducer elements. Using electronic beam steering, three groups of foci (total 39 foci) in cardiac muscle were defined at short, medium and long (20, 40 and 60 mm) radial distances from the transducer surface and at different steering angles from the transducer radial axis. A full range of ultrasound pressure distribution in a volume of 60×80×80 mm³, including esophageal wall, was calculated using a multilayer acoustic wave transmission model for each focus. The corresponding thermal effect in both esophageal wall and cardiac tissue due to the acoustic energy absorption was simulated using the bioheat transfer equation. For short, medium and long (1-, 10-, and 20-second sonications that did not produce thermal lesions in the esophageal wall, the acoustic power ranges needed to achieve a 60° C. maximum temperature in cardiac muscle were 105 W to 727 W, 28 W tol 17 W, 21 W to 79 W, respectively. Similarly, for the same sonications, the acoustic power ranges needed to achieve a 70° C. maximum temperature in cardiac muscle were 151 W to 1044 W, 40 W to 167 W, and 30 W tol 14 W, respectively. A thermal dose in equivalent minutes at 43° C. (denoted T₄₃) was applied to the foci for at least 240 min. The resulting tissue lesion lengths at these foci were 1-6, 3-11, 3-13 mm and 3-15, 5-19, 6-23 mm, respectively. The lesion widths were 1-4, 2-7, 3-9 mm and 3-9, 4-13, 4-17 mm, respectively. The following text describes the studies in greater detail.

Simulation Configuration

The tissue lesions produced by various transesophageal ultrasound fields under different sonication powers and durations were simulated on a computer. The two-dimensional planar ultrasound phased array that was modeled in the studies had a length of 60 mm, a width of 10 mm, produced ultrasound at 1 MHz, and included 2280 transducers having a 0.525 mm inter-element center-to-center spacing arranged as a 1 14×20 grid such that the diagonal and maximum transducer array inter-element center-to-center spacing was less than half a wavelength (0.75 mm at 1 MHz). The planar phased array was capable of steering the beam by proper element phasing and amplitude weighting with respect to the distances from the elements to the focus. The full steering functionality enabled the array to aim the beam and track cardiac tissue motion during sonication. The planar phased array was also modeled to be positioned inside an esophagus, facing the heart, with water filling the space between the transducer and the esophageal wall. The simulation assumed a 4.4 mm esophageal wall thickness and defined the inner and outer surfaces of the esophageal wall as being the surfaces that were in contact with water and cardiac muscle. Each of the surfaces of the esophageal wall was interpolated from seven arcs that were evenly distributed along z-axis. Each arc was constructed as either a half circle having a 10 mm radius for the outer surface or a 5.6 mm radius for the inner surface and being centered on the z-axis. Sixty evenly spaced points were randomly selected along the arc such that the distances from the points to the z-axis varied randomly by a distance up to ±2 mm. The variation of the points deformed the half-circle arc to an irregular arc centered at the z-axis, which was constructed by a spline interpolation on the points. The whole outer surface was then linearly interpolated from the seven smooth arcs centered at the z-axis. In this way, up to 2 mm curvature variations were added onto the esophageal wall surfaces to approximate the uneven surfaces.

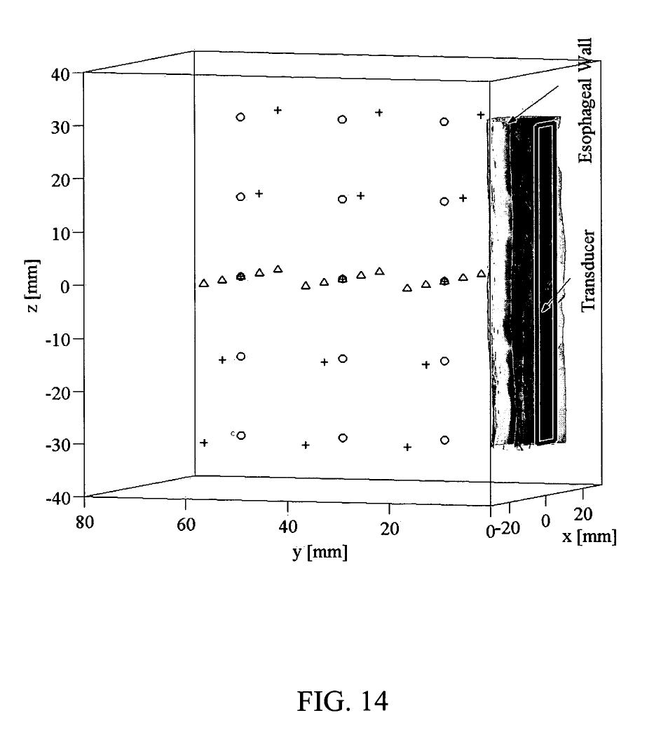

FIG. 14 shows a diagram of the relative positions of the transducer array, the esophageal wall, the cardiac muscle and the locations (referred to as foci) on the cardiac muscle at which tissue lesions were produced. Three groups of foci (39 foci in total) were placed at short, medium and long (20, 40 and 60 mm) radial ranges and different steering angles from the transducer surface. A first group of foci contained 15 foci (focus numbers 1 to 15, shown as circles in FIG. 14) in the x=0 plane. A second group of foci contained 15 foci (focus numbers 7, 8, 9, and 16 to 27, shown as triangles in FIG. 14) in the z=0 plane. A third group of foci contained 15 foci (focus numbers 7, 8, 9, and 28 to 39, shown as plus signs in FIG. 14) in a slanted plane between x=0 and z=0 planes. The three planes intersect at y-axis and the three groups shared three common foci (focus numbers 7, 8 and 9, shown as overlaying of circles, triangles and plus signs in FIG. 14). The three groups share the same three foci in the y-axis. Table 1 shows the coordinates of each of the foci.

TABLE 1

| focus | coordinates | focus | coordinates | focus | coordinate | focus | coordinate |
|---|---|---|---|---|---|---|---|
| number | [x, y, z] mm | number | [x y z] mm | number | [x y z] mm | number | [x y z] mm |
| 1 | [0, 20, 30] | 11 | [0, 40, −15] | 21 | [10 60 0] | 31 | [10 20 15] |
| 2 | [0, 40, 30] | 12 | [0, 60, −15] | 22 | [−10 20 0] | 32 | [10 40 15] |
| 3 | [0, 60, 30] | 13 | [0, 20, −30] | 23 | [−10 40 0] | 33 | [10 60 15] |
| 4 | [0, 20, 15] | 14 | [0, 40, −30] | 24 | [−10 60 0] | 34 | [−10 20 −15] |
| 5 | [0, 40, 15] | 15 | [0, 60, −30] | 25 | [−20 20 0] | 35 | [−10 40 −15] |
| 6 | [0, 60, 15] | 16 | [20 20 0] | 26 | [−20 40 0] | 36 | [−10 60 −15] |
| 7 | [0, 20, 0] | 17 | [20 40 0] | 27 | [−20 60 0] | 37 | [−20 20 −30] |
| 8 | [0, 40, 0] | 18 | [20 60 0] | 28 | [20 20 30] | 38 | [−20 40 −30] |
| 9 | [0, 60, 0] | 19 | [10 20 0] | 29 | [20 40 30] | 39 | [−20 60 −30] |
| 10 | [0, 20, −15] | 20 | [10 40 0] | 30 | [20 60 30] | | |

The simulation evaluated the near field heating in the esophageal wall that was caused by absorbed acoustic intensity. To accomplish this, a full range of acoustic pressure field distributions were calculated in a three-dimensional orthogonal grid of field points for each focus. The pressure field distribution calculated by the simulation spanned from −40 to 40 mm in the x-axis, 0 to 80 mm in the y-axis, and −40 to 40 mm in the z-axis. The same three-dimensional grid was also used in a finite difference thermal simulation.

Transesophageal Acoustic Field Calculation

Continuous-wave sonications were modeled in the computer simulation studies. The transesophageal ultrasound pressure fields in cardiac muscle were calculated with a multilayer acoustic wave transmission model that considered both attenuation in a tissue layer and refraction at a curved tissue layer interface. In this model, a tissue layer interface was partitioned into planar rectangular mesh patches that were small enough (about a quarter wavelength in dimension) to be treated as simple sources. The working variables were the particle normal velocities on these patches. The particle normal velocity at a field point in front of a tissue layer interface was calculated using a Rayleigh-Sommerfeld surface integral over the simple sources on the tissue interface. Each simple source was assumed to be only radiating in its forward half space to model the ultrasound non-illuminating area obstructed by tissue geometry. The refracted particle normal velocities at each tissue layer interface were approximated using Snell's law on the planar patches. To simulate the non-transmitting situation, a total possible reflection was considered by calculating the incident angle of each acoustic beam from any simple source to the current patch. For a multilayer problem, the propagation-refraction processes at the multiple interfaces cascade one layer after another to produce the transmitted acoustic field from the curved tissue layers.

The transducer surface was treated as a simple acoustic source interface that radiated acoustic waves. The waves then propagated through the water layer, the inner esophageal wall, the esophagus layer and the outer esophageal wall and into the cardiac muscle. The tissue interface partitioning mesh size was 0.5×0.5 mm² in the study. The acoustic properties of the media are listed below in Table 2.

TABLE 2

| | | | acoustic | specific | thermal | blood |
|---|---|---|---|---|---|---|
| | | | attenuation | heat | conduc- | perfusion |
| | density | speed of | coefficient at 1 | capacity | tivity | rate |
| medium | (kg/m³) | sound (m/s) | MHz (Np/m) | (J · kg/K) | (W/m/K) | (kg/m³/s) |
| water | 1500 | 1000 | 2.88 × 10⁻⁴ | 4180 | 0.615 | 0 |
| esophagus | 1650 | 1040 | 7 | 3200 | 0.5 | 14.2 |
| cardiac muscle | 1572 | 1060 | 4.1 | 3720 | 0.537 | 7.1 |
| blood | 1030 | — | — | 3620 | — | — |

The ratio of the acoustic pressure to the associated normal particle velocity in a medium is the specific acoustic impedance of the medium. Ignoring non-linearity, the acoustic pressure in a small (about a quarter wavelength in dimension) patch can be obtained from the product of the particle normal velocity and the specific acoustic impedance of the tissue. A reported human esophagus speed of sound measurement could not be found in published literature. Therefore, the speed-of-sound value corresponds to a speed-of-sound measurement of a pig esophagus sample using a scanning laser acoustic microscope.

Tissue Coagulation Simulation

The temporal profile of tissue temperature spatial distribution (T(x, y, z, t)) during ultrasound sonication was modeled using the Pennes bioheat transfer equation:

$${ρ}_{t} {C}_{t} \frac{\partial T ( x , y , z , t )}{\partial t} = ∇ \cdot [ {k}_{t} ∇ T ( x , y , z , t ) ] - {WC}_{b} [ T ( x , y , z , t ) - {T}_{a} ] + \frac{α {\vert p ( x , y , z ) \vert}^{2}}{{ρ}_{t} c} \qquad Eq . 1$$

where ρₜ, Cₜ and kₜ are the density, specific heat capacity, and thermal conductivity of the tissue, C_{b} and W are the specific heat capacity and the perfusion rate of the blood. The variables a, c, p(x, y, z) represent acoustic pressure attenuation, the speed of sound of the tissue, and the acoustic pressure amplitude in the tissue. The variable Tₐ is the body temperature (37° C.). The first, second, and third term in the right hand side of Equation 1 simulate the heat conduction in tissue, heat loss due to blood perfusion and energy absorption from the acoustic field, respectively. The third term is the specific absorption rate (SAR) as a measure of energy absorption rate from external energy sources. The pressure amplitude p(x, y, z) was calculated in the ultrasound field simulation. The thermal properties of the media are listed in Table 2.

The temperature profile (T(x,y,z,t)) obtained by solving Equation 1 was then mapped to a thermal dose in equivalent minutes at 43° C. (T₄₃ (x, y, z)) using Sapareto and Dewey's thermal dose function expressed as:

$$\begin{matrix}{T}_{43} ( x , y , z ) = {\int }_{0}^{t} {R}^{43 - T ( x , y , z , t )} ⅆ t , \\ where R = \{ \begin{matrix}0.5 & ( T ( x , y , z , t ) \leq 43 ° C . ) \\ 0.25 & ( T ( x , y , z , t ) > 43 ° C . )\end{matrix}\end{matrix} \qquad Eq . 2$$

Tissue was considered necrotic when T₄₃(x,y,z) exceeded 240 minutes in the simulation volume.
Numerical Implementation

The acoustic pressure amplitude spatial distribution (p(x, y, z)) in a volume of 60×80×80 mm was calculated for each of the 39 foci using the multilayer acoustic wave transmission model. The pressure distribution in the volume was sampled with a three-dimensional rectangular grid of field points. To determine the array element phase necessary for forward transesophageal beam steering, a reverse transesophageal propagation process, in which a point source was radiating at the desired focus, was first simulated using the multilayer transmission model to obtain the reverse complex pressure at each element. The conjugated phase of the reverse complex pressure was fed to each element in the forward transesophageal propagation. This ensured the necessary phase compensation for both beam steering and phase aberration correction caused by esophageal wall. The source intensity of each element was weighted by the distance between the element and the focus so that the propagated wave amplitude at the focus from all elements would be the same.

Because the transducer array had a 60 mm length along the z-axis and a 10 mm width along the x-axis, the beam width along the z-axis was narrower than that along the x-axis. The spatial grid spacing had to be very small to accurately capture the beam pressure amplitude profile along the z-axis. Furthermore, the pressure distribution (p(x, y, z)) was used to calculate the specific absorption rate (SAR=a|p(x, y, z)|²/ρC) in thermal simulation. Therefore, the narrow beam width in z-axis also facilitated the use of a fine grid in the thermal simulation domain to ensure spatial convergence of T(x, y, z, t) using a finite difference scheme. However, a fine spatial sampling of the pressure amplitude would have led to an expensive calculation cost to obtain the p(x, y, z) in the given volume. To reduce the computational cost and yet to maintain a fine resolution of acoustic pressure field for the following thermal simulation, a non-uniform three-dimensional grid was used in calculating the p(x, y, z) in the given volume, with the smallest spacing in the focal region and the largest spacing in the marginal region outside of the beam. Several small spatial grid spacings in the focal region (0.5 mm, 0.25 mm and 0.125 mm) were examined in the thermal simulation to evaluate spatial convergence of the calculated temperature field.

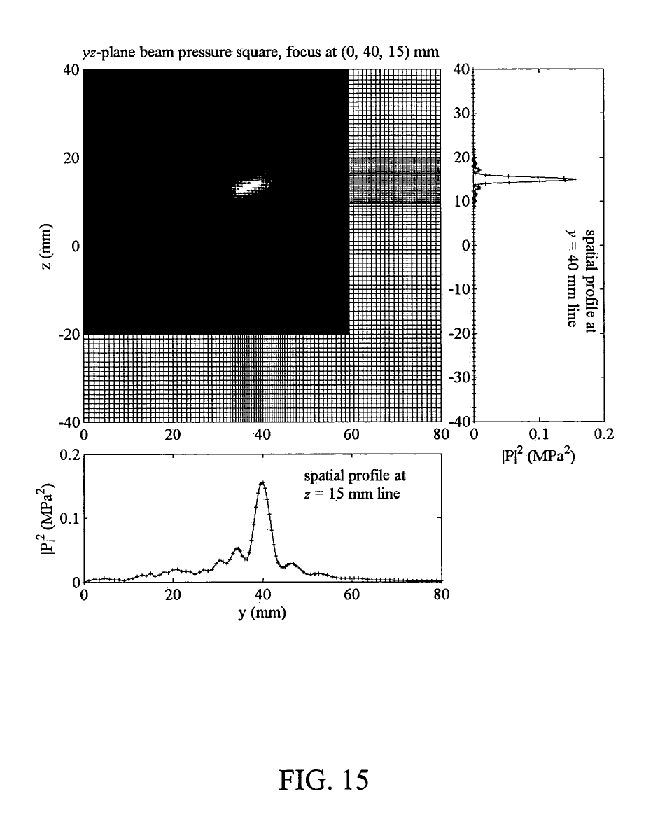

FIG. 15 shows an example of a non-uniform spatial grid and the simulated ultrasound beam in the yz-plane. The “+” marks in the beam spatial profile plots represent grid sampling location. A 0.5-mm grid spacing was sufficient to capture spatial temperature change across the focal region along the y-axis, while a 0.25-mm grid spacing was effective for capturing the spatial temperature change across the focal region along the z-axis. Based on the spatial temperature field convergence test, the grid spacing along the z-axis varied from 0.25 mm to 1 mm, and the grid spacing along the x-axis and along the y-axis varied from 0.5 mm to 1 mm in the study. The grid spacing change took a smooth transition from the focal region to the marginal region to ensure that the spatial derivative in Equation 1 was accurate and stable.

The bioheat equation (Equation 1) was solved using a finite difference scheme in Cartesian coordinates to obtain T(x, y, z, t). Because of the minor differences in their values, the inhomogeneity of thermal conductivity kₜ in different media was ignored for implementation simplicity. Due to the non-uniform grid, a modified spatial derivative operator using a central differencing scheme was adopted instead of its conventional counterpart for uniform grid. The resulting discrete equation of Equation 1 is

$${T}_{i , j , k}^{n + 1} = {T}_{i , j , k}^{n} + \frac{Δ t}{{ρ}_{{t}_{i , j , k}} {C}_{{t}_{i , j , k}}} \times \{ \begin{matrix}{k}_{i , j , k} [ P ( {T}_{i , j , k}^{n} , {x}_{i} ) + Q ( {T}_{i , j , k}^{n} , {y}_{i} ) + R ( {T}_{i , j , k}^{n} , {z}_{i} ) ] - \\ {W}_{i , j , k} {C}_{{b}_{i , j , k}} [ {T}_{i , j , k}^{n} - {T}_{a} ] + \frac{{α}_{i , j , k} {\vert {p}_{i , j , k} \vert}^{2}}{{ρ}_{{t}_{i , j , k}} {c}_{i , j , k}}\end{matrix} \} \qquad Eq . 3$$

with the discrete operators P(·), Q(·) and R(·) being defined as:

$$P ( {T}_{i , j , k}^{n} , {x}_{i} ) = ( \frac{{T}_{i - 1 , j , k}^{n} - {T}_{i , j , k}^{n}}{{x}_{i - 1}^{n} - {x}_{i}^{n}} - \frac{{T}_{i , j , k}^{n} - {T}_{i + 1 , j , k}^{n}}{{x}_{i}^{n} - {x}_{i + 1}^{n}} ) / ( \frac{{x}_{i - 1}^{n} + {x}_{i}^{n}}{2} - \frac{{x}_{i}^{n} + {x}_{i + 1}^{n}}{2} ) \\ Q ( {T}_{i , j , k}^{n} , {y}_{j} ) = ( \frac{{T}_{i , j - 1 , k}^{n} - {T}_{i , j , k}^{n}}{{y}_{j - 1}^{n} - {y}_{j}^{n}} - \frac{{T}_{i , j , k}^{n} - {T}_{i , j + 1 , k}^{n}}{{y}_{j}^{n} - {y}_{j + 1}^{n}} ) / ( \frac{{y}_{j - 1}^{n} + {y}_{j}^{n}}{2} - \frac{{y}_{j}^{n} + {y}_{j + 1}^{n}}{2} ) \\ R ( {T}_{i , j , k}^{n} , {z}_{k} ) = ( \frac{{T}_{i , j , k - 1}^{n} - {T}_{i , j , k}^{n}}{{z}_{k - 1}^{n} - {z}_{k}^{n}} - \frac{{T}_{i , j , k}^{n} - {T}_{i , j , k + 1}^{n}}{{z}_{k}^{n} - {z}_{k + 1}^{n}} ) / ( \frac{{z}_{k - 1}^{n} + {z}_{k}^{n}}{2} - \frac{{z}_{k}^{n} + {z}_{k + 1}^{n}}{2} ) \qquad Eq . 4$$

where n is the discrete time step, i, j, k are the indices for the nonuniform grids in the x-, y- and z-axes and take all the integer values between the second and the second to the last indices. The specific-absorption rate (SAR) term in Equation 3 used an average value in the voxel by taking arithmetic mean among the |p_{i,j,k}|² an d its neighboring values. As a reasonable approximation when dealing with a large volume in which the thermal source was far away from the boundaries, Neumann boundary conditions

$$( \frac{\partial T}{\partial n} = 0 )$$

were set on the tissue volume surfaces. The temporal derivative was implemented using a forward differencing scheme in Equation 3. Corresponding with the smallest spatial grid spacing aforementioned, the time step size was chosen as 0.05 s for a stable finite difference thermal simulation.

Before each sonication, a pre-cooling phase was used to lower the initial temperature of the esophageal wall and to reduce the risk of thermal damage in esophagus. In the pre-cooling phase, 20° C. degassed water was filled in between the transducer and the esophageal wall as coupling medium. The initial water temperature and the tissue temperature were 20° C. and 37° C., after which the temperature field evolved to its steady state without external sonication (SAR=0 in Equation 1). Approximately 190 seconds later, the water-tissue system reached its steady condition as defined by a maximum temperature change between two consecutive time steps that was less than 0.01° C. At this steady condition, the mean temperatures in the inner and outer surfaces of the esophageal wall were 20° C. and 32.5° C., respectively. The steady condition was then used as the initial condition for a following sonication.

Results

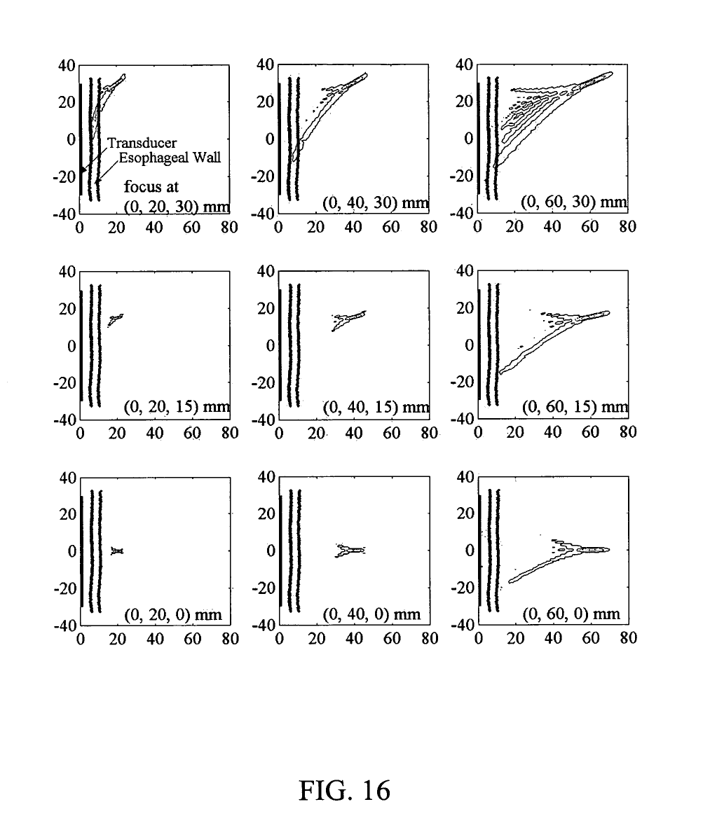

FIG. 16 shows nine plots of the yz-plane squared pressure-amplitude contours determined for the acoustic focal beams corresponding to focus numbers 1 to 9 in Table 1. The horizontal and vertical axes of the plots are y- and z-axes in millimeter units, respectively. The value difference between two adjacent contour lines is 20% of the peak pressure square value of the field.

Transesophageal focal beam steering was achieved in a wide range of the field. The near-field squared pressure amplitudes increased when the beam steering angle increased. The average value that the foci shifted away from their intended focal locations was 0.9±0.7 mm for the 39 foci.

Three sonication durations of 1-, 10- and 20-seconds were adopted to simulate the short, medium and long ultrasound exposure times. The peak temperature at each focus was set as 60° C. or at 70° C. at the end of each sonication. The cooling times for the 1-, 10-, and 20-second sonications were adequately set as 24, 40 and 50 seconds to allow for tissue temperature dropping back close to 37° C. The simulated lesions at the 39 foci at 1-, 10-, and 20-second sonications to reach a 60° C. or 70° C. peak temperature were examined, with a total of 234 simulated lesions.

The disadvantage of using an intra-cavity planar probe for thermal ablation is the higher-than-normal transducer power requirement needed to achieve high enough focal intensity for tissue coagulation. Consequently, potential thermal damage to the intervening tissue layer could occur due to the proximity between the probe and the cavity wall. This may spatially compromise the safe thermal ablation zone for the planar phase array. To evaluate the safety of these sonications to patients, the thermal dose accumulation inside esophageal wall was calculated. Sonications that did not cause the thermal dose in equivalent minutes at 43° C. (T₄₃) to be greater than 5 minutes in the esophageal wall are referred to as “safe” sonications. Sonications that produced a T₄₃ greater than or equal to 5 minutes in the esophageal wall are referred to as “unsafe” sonications. The 5-minute T₄₃ threshold imposed a conservative safety criterion for thermal damage estimation.

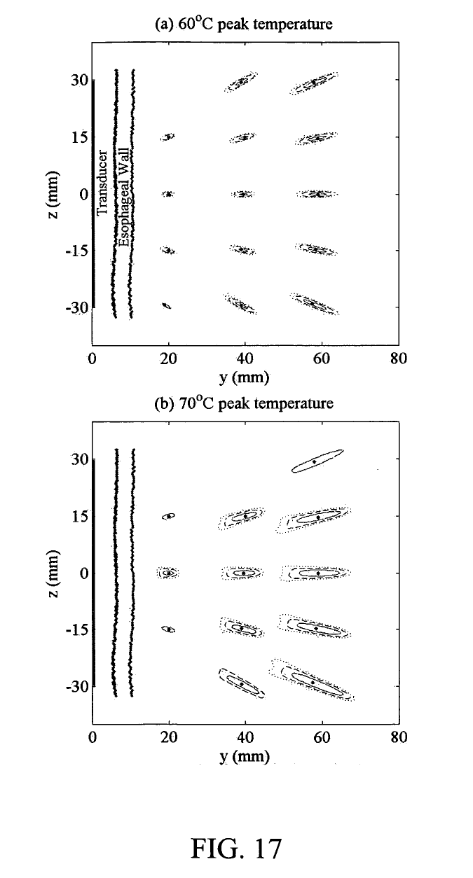

The 15 foci of the first group, having focus numbers 1 to 15, were in the x=0 plane. FIG. 17 shows plots of simulated yz-plane contours that result from sonicating the first group of foci at peak temperatures of 60° C. and 70° C. and at a thermal dose in equivalent minutes at 43° C. (T₄₃=240 min). The solid, dashed and dotted contours represent 1-, 10- and 20-second sonication durations. These sonications were safe sonications. Depending on the steering angle and sonication time, the safety limit thermal dose (T₄₃≧5 min) was reached in esophageal wall for other simulated lesion volumes. For each focus at the same peak temperature, the tissue lesion size enlarged when the sonication duration increased. In sonications at the same peak temperature, the tissue lesion size enlarged when the steering angle increased. The lesion sizes at the 70° C. peak temperature were larger than those at the 60° C. peak temperature.

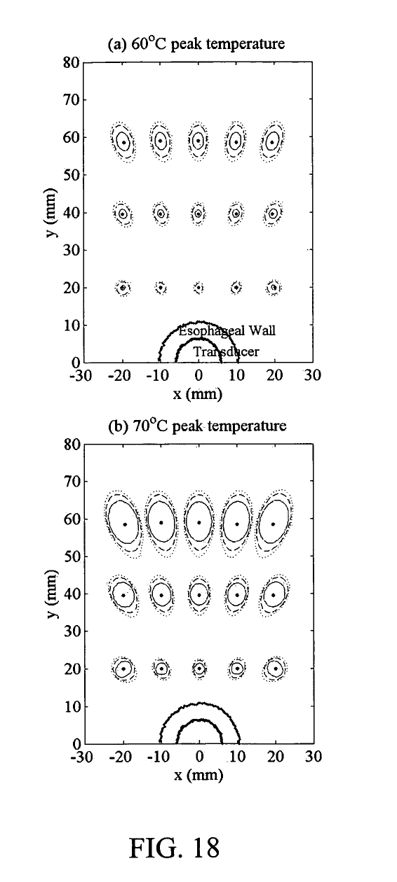

The 15 foci of the second group (focus numbers 7, 8, 9, and 16 to 27) were in the z=0 plane. FIG. 18 shows plots of simulated xy-plane contours that result from sonicating the second group of foci at peak temperatures of 60° C. and 70° C. and at a thermal dose (T₄₃) of 240 min. The solid, dashed and dotted contours represent 1-, 10- and 20-second sonication durations. All of these sonications in z=0 plane were safe sonications.

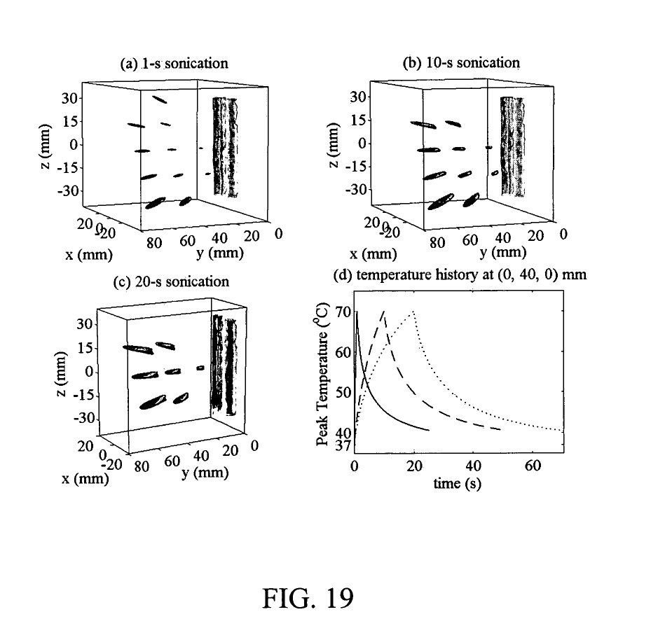

The 15 foci of group 3 were in a slanted plane between x=0 and z=0 planes, spanning a slanted slice in the cardiac muscle. FIG. 19 shows plots of simulated isosurfaces that result from sonicating the third group of foci at a peak temperature of 70° C. with a thermal dose (T₄₃) of 240 min over 1-, 10- and 20-second safe sonication durations. FIG. 19 also shows a plot of the temperature history at 0-, 40-, 0-mm for the 1-, 10- and 20-second safe sonications.

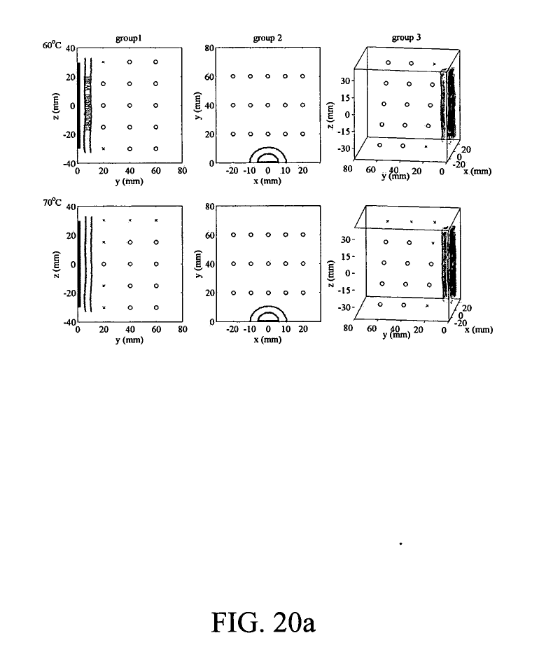

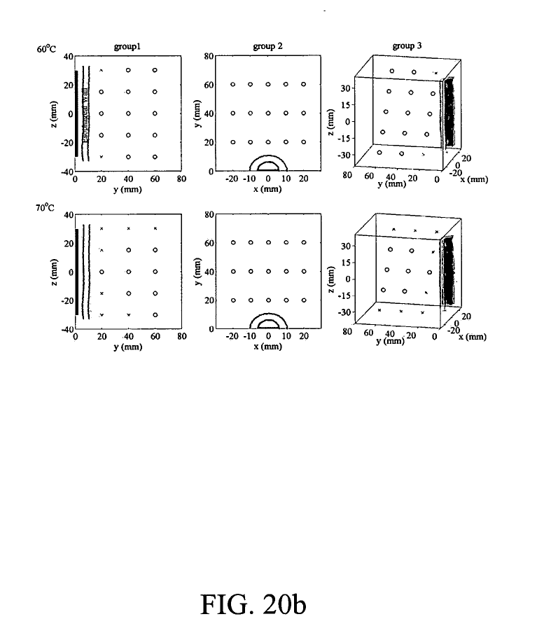

FIGS. 20a and 20b show plots that summarize the occurrence of the 10- and 20-second safe and unsafe sonications in the three foci groups at peak temperatures of 60° C. and 70° C., respectively. The occurrences of safe sonications are marked by an “o” and the occurrences of unsafe sonications are marked by an “x”. The safe sonication zone was not symmetric due to the surface curvature variations added in constructing the esophageal wall. The range of the beam steering angle and distance at which safe sonications could be achieved were more limited for the simulations with higher peak temperatures and longer sonication times than for the simulations with the lower peak temperature and shorter sonication times.

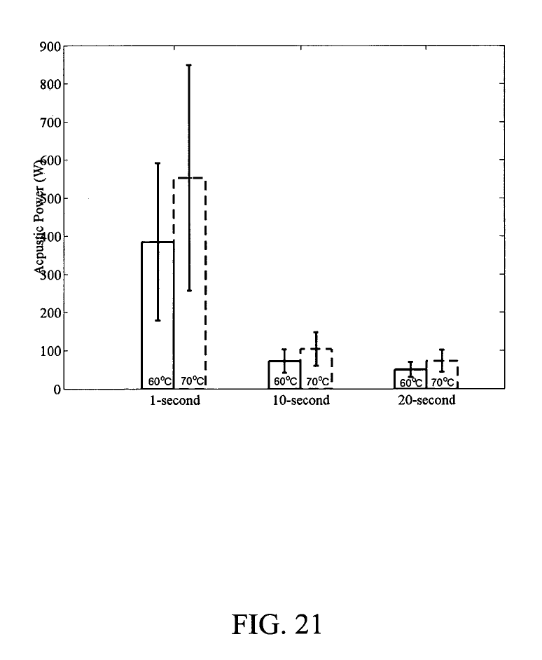

FIG. 21 shows a plot of the average transducer acoustic powers that produced peak temperature of 60° C. and 70° C. peak at the foci for the 1-, 10- and 20-second safe sonications.

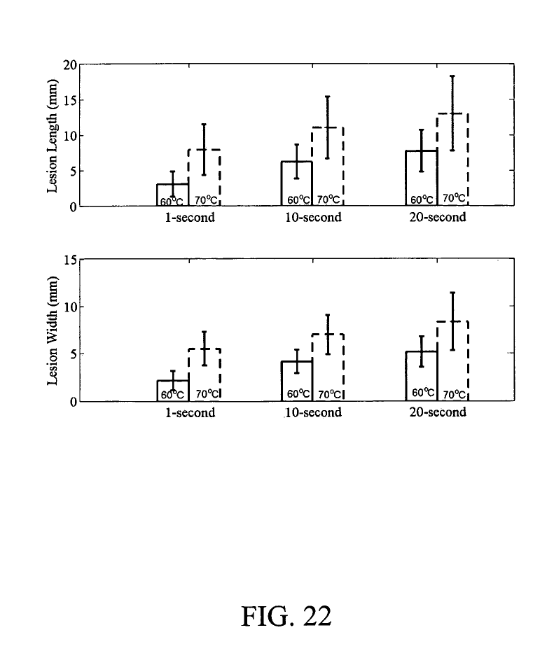

FIG. 22 shows a plot of average lengths and widths of the lesions that resulted from the safe sonications described in FIG. 21. These sonications were simulated at a thermal dose (T₄₃) was applied for at least 240 min. Table 3 lists the transducer acoustic power ranges needed to achieve 60° C. and 70° C. peak temperatures at the foci for 1-, 10-and 20-second safe sonications and the corresponding lesion length and width ranges.

TABLE 3

| | | | |
|---|---|---|---|
| Sonication Duration | Acoustic Power | Lesion Length | Lesion Width |
| (s) | (W) | (mm) | (mm) |

60° C. peak temperature

| | | | |
|---|---|---|---|
| 1 | 105-727 | 0.7-6.4 | 0.6-3.9 |
| 10 | 28-117 | 2.7-10.8 | 2.0-7.0 |
| 20 | 21-79 | 3.3-13.4 | 2.7-9.3 |

70° C. peak temperature

| | | | |
|---|---|---|---|
| 1 | 151-1044 | 2.9-14.5 | 2.8-8.7 |
| 10 | 40-167 | 4.6-19.1 | 3.6-13.2 |
| 20 | 30-114 | 5.6-23.0 | 4.0-16.9 |

Table 4 lists the maximum, minimum and mean peak pressure amplitude at the foci for the 1-, 10-, 20-second safe sonications at 60° C. and 70° C. peak temperatures.

TABLE 4

| | | | |
|---|---|---|---|
| Sonication | Maximal Pressure | Minimal Pressure | Mean Pressure |
| Duration (s) | (MPa) | (MPa) | (MPa) |

60° C. peak temperature

| | | | |
|---|---|---|---|
| 1 | 7.7 | 6.4 | 6.8 ± 0.4 |
| 10 | 4.0 | 2.6 | 3.1 ± 0.4 |
| 20 | 3.5 | 2.1 | 2.6 ± 0.4 |

70° C. peak temperature

| | | | |
|---|---|---|---|
| 1 | 9.3 | 7.7 | 8.2 ± 0.5 |
| 10 | 4.8 | 3.1 | 3.7 ± 0.5 |
| 20 | 4.7 | 2.6 | 3.1 ± 0.5 |

Discussion

At 1 MHz, the simulated planar two-dimensional phased array (60×10 mm²) was able to steer and focus its beam through esophageal wall into cardiac muscle through a wide range of angles. By varying sonication duration and power, the array produced a thermal dose that was high enough to cause tissue necrosis of different sizes. Therefore, on it was feasible to use a two-dimensional planar ultrasound phased array for transesophageal cardiac thermal ablation.

The esophagus offers a convenient ultrasound window to the heart, particularly, the back structures, such as the atria. Such proximity makes the proposed transesophageal ultrasound ablation technique promising since the esophagus tissue layer induces minimal distortion of the wave. The flexible transesophageal three-dimensional beam steering can produce continuous thermal lesions by properly planning ablation locations. Furthermore, such a flexible three-dimensional beam steering capability enables the motion of a beating heart to be tracked during sonication.

Ultrasound pressure greater than a certain threshold may cause acoustic cavitation in biological tissues. The possibility of inertial cavitation under these power levels for the three sonication durations was examined by comparing the peak pressure at the foci with the cavitation pressure threshold in muscle in vivo. The peak pressure values for all the 10- and 20-second sonications (shown in Table 4) were below the cavitation threshold in dog muscle (5.3 MPa at 1 MHz). The peak pressure values for all the 1-second sonications (shown in Table 4) were greater than the cavitation threshold because more acoustic energy was needed to coagulate tissue in a very short time. One should, however, note that there are no cavitation threshold measurements for cardiac tissue. The cavitation phenomena can be utilized to enhance tissue heating and may also be useful for cardiac ablation. The cavitation phenomena, however, was not simulated in this study. To achieve high enough peak temperature for a short sonication time while suppressing cavitation, a higher operating frequency has to be used to raise the cavitation threshold. Or, the sonication duration must be long enough to allow the acoustic pressure, which is lower than cavitation threshold, to slowly produce a thermal lesion.

Power requirement is a practical concern when designing a phased array for thermal ablation. The proposed array size in this study was 60×10 mm². The acoustic intensity on an array surface is high when thermal lesions are to be produced rapidly. For example, the transducer acoustic power requirements for achieving 70° C. peak temperature with 1-, 10- and 20-second sonication at foci (0, 40, 0) mm were 377, 80, and 58 W and corresponded to acoustic intensities of about 63, 13, 10 W/cm² on the transducer surface, respectively. The planar transducer array size can be increased to increase the focal pressure gain and add transducer surface area to reduce the power requirement. The human esophagus is about 25 mm in diameter. The proposed transducer array width was only 10 mm in this study; however, the transducer array width could be enlarged to reduce the power requirement of the array. The corresponding acoustic powers for a larger transducer (60×20 mm^{2) were} 261, 60, 45 W (acoustic intensity on the transducer surface about 22, 5, 4 W/cm²), respectively. These acoustic power outputs are within the reach of current transducer array technology. With these arrays, however, the number of transducer elements becomes an issue in RF power amplifier design and channel wiring. There are tradeoffs between the transducer element size, sonication duration and acoustic power.

A number of embodiments of the invention have been described. Nevertheless, it should be understood that various modifications may be made without departing from the spirit and scope of the invention. Other embodiments are within the scope of the following claims.

What is claimed is:

1. A system for performing ablation of target tissue, the system comprising:
an ultrasound phased array having a plurality of ultrasonic transducers;
drive circuitry coupled to the ultrasonic transducers, the drive circuitry configured to generate signals that cause the ultrasonic transducers to focus ultrasound radiation at the target tissue;
a diagnostic system configured to collect diagnostic data percutaneously, the diagnostic data being indicative of a condition of the target tissue; and
computational circuitry interfaced to the drive circuitry and to the diagnostic system, the computational circuitry configured to control the drive circuitry based on the diagnostic data.

2. The system of claim 1, wherein the ultrasound phased array is configured for placement in a patient's esophagus.

3. The system of claim 1, wherein the ultrasound phased array is two-dimensional.

4. The system of claim 1, wherein the drive circuitry comprises multi-channel radio-frequency drivers.

5. The system of claim 1, wherein the diagnostic system comprises an imaging system and the diagnostic data comprises an image of the target tissue.

6. The system of claim 5, wherein the imaging system comprises one of: a magnetic resonance imaging (MRI) system, an ultrasound imaging system, a computed tomography imaging system, an x-ray imaging system, and a positron-emission tomography imaging system.

7. The system of claim 1, wherein the diagnostic system comprises a temperature monitoring system.

8. The system of claim 7, wherein the temperature monitoring system comprises an MRI system.

9. The system of claim 1, wherein the diagnostic system is configured to collect the diagnostic data in real-time.

10. A method for performing ablation of target tissue, the method comprising:
identifying a target location of the target tissue from an image of the target tissue;
focusing ultrasound radiation from an ultrasound phased array at the target location;
collecting diagnostic data percutaneously, the diagnostic data being indicative of a condition of the target tissue; and
controlling a characteristic of the ultrasound radiation based on the diagnostic data such that the ultrasound radiation ablates the target tissue without damaging surrounding tissue.

11. The method of claim 10, wherein identifying a target location of a target tissue comprises determining a target location in relation to a periodic triggering event.

12. The method of claim 10, wherein identifying a target location of the target tissue comprises determining the target location in real-time.

13. The method of claim 10, further comprising transforming coordinates of the image to coordinates of the ultrasound phased array.

14. The method of claim 11, wherein focusing ultrasound radiation comprises focusing ultrasound radiation at the target location for a predefined period of time in response to detecting the triggering event.

15. The method of claim 14, wherein detecting the triggering event comprises detecting a heartbeat.

16. The method of claim 10, wherein controlling a characteristic of the ultrasound radiation comprises selecting at least one of a phase, frequency, and power of the ultrasound radiation.

17. The method of claim 10, wherein collecting diagnostic data further comprises acquiring temperature data that is indicative of ablation.

18. A computer readable medium having, stored thereon, software for performing ablation of target tissue, the software comprising instructions for causing a computer to:
identify a target location of the target tissue from an image of the target tissue;
focus ultrasound radiation from an ultrasound phased array at the target location;
collect diagnostic data percutaneously, the diagnostic data being indicative of a condition of the target tissue; and
control a characteristic of the ultrasound radiation based on the diagnostic data such that the ultrasound radiation ablates the target tissue without damaging surrounding tissue.

19. The computer readable medium of claim 18, wherein the software further comprises instructions that cause the computer to transform coordinates of the image to coordinates of the ultrasound phased array.

20. The computer readable medium of claim 18, wherein the software further comprises instructions that cause the computer to acquire temperature data that is indicative of ablation.
